(19) United States
(12) Patent Application Publication
(10) Pub. No.: US 2019/0192799 A1
(43) Pub. Date: Jun. 27, 2019

(54) DELIVERY OF A PRESSURE SUPPORT THERAPY

(71) Applicant: KONINKLIJKE PHILIPS N.V., EINDHOVEN (NL)

(72) Inventors: DANIEL STEED, NORTH HUNTINGDON, PA (US); ROBERT WILLIAM BAIKO, PITTSBURGH, PA (US); RICHARD ANDREW SOFRANKO, FINLEYVILLE, PA (US); JONATHAN SAYER GRASHOW, PITTSBURGH, PA (US)

(21) Appl. No.: 16/224,844

(22) Filed: Dec. 19, 2018

Related U.S. Application Data
(60) Provisional application No. 62/609,353, filed on Dec. 22, 2017.

(51) Int. Cl. A61M 16/06; A61B 5/00

(52) U.S. Cl. CPC A61M 16/0622; G16H 20/40; A61B 5/4818

(57) ABSTRACT

A method of improving delivery of a pressure support therapy provided by a pressure support device to a patient via a patient interface device includes begins by receiving current data regarding details of one or more of the patient and the delivery of the pressure support therapy. The current data is then compared to stored data. If a change is determined between the current data and the stored data which is indicative of a poor mask fitment, an indication is provided of a need for a new patient interface device fitting for the patient.

### CROSS-REFERENCE TO RELATED APPLICATIONS

This patent application claims the priority benefit under 35 U.S.C. § 119(e) of U.S. Provisional Application No. 62/609,353, filed on Dec. 22, 2017, the contents of which are herein incorporated by reference.

### BACKGROUND OF THE INVENTION

### 1. Field of the Invention

The present invention pertains to methods of improving delivery of a pressure support therapy. The present invention also pertains to systems for improving delivery of a pressure support therapy.

### 2. Description of the Related Art

Many individuals suffer from disordered breathing during sleep. Sleep apnea is a common example of such sleep disordered breathing suffered by millions of people throughout the world. One type of sleep apnea is obstructive sleep apnea (OSA), which is a condition in which sleep is repeatedly interrupted by an inability to breathe due to an obstruction of the airway; typically the upper airway or pharyngeal area. Obstruction of the airway is generally believed to be due, at least in part, to a general relaxation of the muscles which stabilize the upper airway segment, thereby allowing the tissues to collapse the airway. Another type of sleep apnea syndrome is a central apnea, which is a cessation of respiration due to the absence of respiratory signals from the brain's respiratory center. An apnea condition, whether obstructive, central, or mixed, which is a combination of obstructive and central, is defined as the complete or near cessation of breathing, for example a 90% or greater reduction in peak respiratory airflow.

Those afflicted with sleep apnea experience sleep fragmentation and complete or nearly complete cessation of ventilation intermittently during sleep with potentially severe degrees of oxyhemoglobin desaturation. These symptoms may be translated clinically into extreme daytime sleepiness, cardiac arrhythmias, pulmonary-artery hypertension, congestive heart failure and/or cognitive dysfunction. Other consequences of sleep apnea include right ventricular dysfunction, carbon dioxide retention during wakefulness, as well as during sleep, and continuous reduced arterial oxygen tension. Sleep apnea sufferers may be at risk for excessive mortality from these factors as well as by an elevated risk for accidents while driving and/or operating potentially dangerous equipment.

Even if a patient does not suffer from a complete or nearly complete obstruction of the airway, it is also known that adverse effects, such as arousals from sleep, can occur where there is only a partial obstruction of the airway. Partial obstruction of the airway typically results in shallow breathing referred to as a hypopnea. A hypopnea is typically defined as a 50% or greater reduction in the peak respiratory airflow. Other types of sleep disordered breathing include, without limitation, upper airway resistance syndrome (UARS) and vibration of the airway, such as vibration of the pharyngeal wall, commonly referred to as snoring.

It is well known to treat sleep disordered breathing by applying a continuous positive air pressure (CPAP) to the patient's airway. This positive pressure effectively “splints” the airway, thereby maintaining an open passage to the lungs. It is also known to provide a positive pressure therapy in which the pressure of gas delivered to the patient varies with the patient's breathing cycle, or varies with the patient's breathing effort, to increase the comfort to the patient. This pressure support technique is referred to as bi-level pressure support, in which the inspiratory positive airway pressure (IPAP) delivered to the patient is higher than the expiratory positive airway pressure (EPAP). It is further known to provide a positive pressure therapy in which the pressure is automatically adjusted based on the detected conditions of the patient, such as whether the patient is experiencing an apnea and/or hypopnea. This pressure support technique is referred to as an auto-titration type of pressure support, because the pressure support device seeks to provide a pressure to the patient that is only as high as necessary to treat the disordered breathing.

Pressure support therapies as just described involve the placement of a patient interface device including a mask component having a soft, flexible sealing cushion on the face of the patient. The mask component may be, without limitation, a nasal mask that covers the patient's nose, a nasal/oral mask that covers the patient's nose and mouth, or a full face mask that covers the patient's face. Such patient interface devices may also employ other patient contacting components, such as forehead supports, cheek pads and chin pads. The patient interface device is typically secured to the patient's head by a headgear component. The patient interface device is connected to a gas delivery tube or conduit and interfaces the pressure support device with the airway of the patient, so that a flow of breathing gas can be delivered from the pressure/flow generating device to the airway of the patient.

In order to optimize treatments as well as patient compliance with such treatments it is important to provide the patient with a well fit mask. As no two patient's faces are exactly the same, the best way to ensure an optimum fit is to provide the patient a custom/semi-custom mask that is sized/designed according to their specific facial geometry. The standard process to generate a custom-fitted mask is to scan the human subject, process the image, and then use an algorithm to generate the custom item (or select an appropriate size for a semi-custom model). While the scan/analyze/select method for custom CPAP masks is realistic, however, there are a few disadvantages. First, the patient must have a scanner or travel to a location that does. Given this drawback, it is likely that the patient will not have frequent updated scans to ensure proper fit, therapy, and satisfaction. A second disadvantage is that this method only takes facial surface geometry into consideration when generating the custom mask while other cues are ignored.

### SUMMARY OF THE INVENTION

As one aspect of the invention, a method of improving delivery of a pressure support therapy provided by a pressure support device to a patient via a patient interface device is provided. The method comprises receiving current data regarding details of one or more of the patient and the delivery of the pressure support therapy; comparing the current data to stored data; determining a change between the current data and the stored data indicative of a poor mask fitment; and providing an indication of a need for a new patient interface device fitting.

The method may further comprise conducting a new patient interface device fitting with the patient and identifying a new patient interface device for the patient. Identifying the new patient interface device for the patient may comprise providing the patient with a specification of the new patient interface device. Identifying the new patient interface device for the patient may comprise providing the patient with the new patient interface device.

Receiving details regarding one or more of the patient and the delivery of the pressure support therapy comprises receiving details regarding a leak score of the patient interface device.

Receiving details regarding one or more of the patient and the delivery of the pressure support therapy may comprise receiving details regarding therapy pressure provided during the pressure support therapy.

Receiving details regarding one or more of the patient and the delivery of the pressure support therapy may comprise receiving details regarding one or more of: weight, heart rate, blood pressure, pulse oximetry, glucose level, body temperature, or recent activity of the patient.

Receiving details regarding one or more of the patient and the delivery of the pressure support therapy may comprise receiving details regarding sleepiness of the patient.

Receiving details regarding one or more of the patient and the delivery of the pressure support therapy may comprise receiving details regarding changes in facial hair of the patient.

Receiving details regarding one or more of the patient and the delivery of the pressure support therapy may comprise receiving details provided by the user in the form of responses to questions. The responses to questions may be provided electronically.

Receiving details regarding one or more of the patient and the delivery of the pressure support therapy may comprise receiving details regarding the patient in the form of images of the patient.

Receiving details regarding one or more of the patient and the delivery of the pressure support therapy may comprise receiving details of the pressure support therapy sent by a communications device which is part of the pressure generating device.

As another aspect of the present invention, a system for use in improving delivery of a pressure support therapy provided to a patient via a patient interface device is provided. The system comprises: a pressure support system having a pressure support device; a processing unit; and a number of input devices, wherein the processing unit is programmed to: receive current data regarding details of one or more of the patient and the delivery of the pressure support therapy from one or more of: the number of input devices and a number of sensors associated with the pressure support device; compare the current data to stored data; determine a change between the current data and the stored data indicative of a poor mask fitment; and provide an indication of a need for a new patient interface device fitting.

The processing unit may be a component of the pressure support device.

The processing unit may be remotely located from the pressure support system.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of a system 2 for improving delivery of a pressure support system according to one particular, non-limiting exemplary embodiment of the disclosed concept;

FIG. 2 is a schematic diagram of a processing unit according to an exemplary embodiment of the disclosed concept; and

FIG. 3 is a flowchart of a method for improving delivery of a pressure support therapy according to one particular, non-limiting exemplary embodiment of the disclosed concept.

### DETAILED DESCRIPTION OF EXEMPLARY EMBODIMENTS

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As used herein, the statement that two or more parts or components “engage” one another shall mean that the parts exert a force against one another either directly or through one or more intermediate parts or components. As used herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

Embodiments of the present invention utilize a holistic approach in identifying a need for an improved fitting CPAP mask for use by a patient in receiving a pressure support therapy.

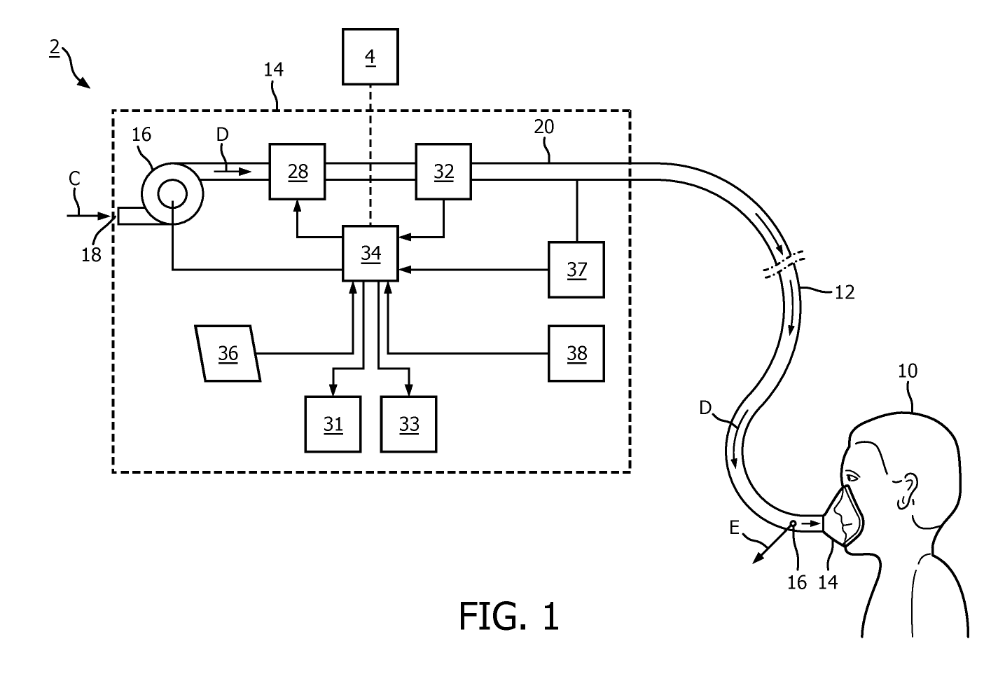

FIG. 1 is a schematic diagram of a system 2 for improving delivery of a pressure support system according to one particular, non-limiting exemplary embodiment in which the present invention may be implemented. System 2 includes a central processing unit 4 and an airway pressure support system 12. Central processing unit 4 includes a processing portion which may be, for example, a microprocessor, a microcontroller or some other suitable processing device, and a memory portion that may be internal to the processing portion or operatively coupled to the processing portion and that provides a storage medium for data and software executable by the processing portion for carrying out methods such as described further below. As discussed below, central processing unit 4 is structured to receive outputs of one or more components of system 2.

Airway pressure support system 12 includes a pressure support device 14 which houses a gas flow generator 16, such as a blower used in a conventional CPAP or bi-level pressure support device. Gas flow generator 16 receives breathing gas, generally indicated by arrow C, from the ambient atmosphere through a filtered air inlet 18 (described in greater detail herein) provided as part of pressure support device 14, and generates a flow of breathing gas therefrom for delivery to an airway of a patient 20 at relatively higher and lower pressures, i.e., generally equal to or above ambient atmospheric pressure. In the exemplary embodiment, gas flow generator 16 is capable of providing a flow of breathing gas ranging in pressure from 3-30 cmH2O. The pressurized flow of breathing gas from gas flow generator 16, generally indicated by arrow D, is delivered via a delivery conduit 22 to a breathing mask or patient interface 24 of any known construction, which is typically worn by or otherwise attached to patient 20 to communicate the flow of breathing gas to the airway of patient 20. Delivery conduit 22 and patient interface device 24 are typically collectively referred to as a patient circuit.

Pressure support system 12 shown in FIG. 1 is what is known as a single-limb system, meaning that the patient circuit includes only delivery conduit 22 connecting patient 20 to pressure support system 12. As such, an exhaust vent 26 is provided in delivery conduit 22 for venting exhaled gases from the system as indicated by arrow E. It should be noted that exhaust vent 26 can be provided at other locations in addition to or instead of in delivery conduit 22, such as in patient interface device 24. It should also be understood that exhaust vent 26 can have a wide variety of configurations depending on the desired manner in which gas is to be vented from pressure support system 12.

The present invention also contemplates that pressure support system 12 can be a two-limb system, having a delivery conduit and an exhaust conduit connected to patient 20. In a two-limb system (also referred to as a dual-limb system), the exhaust conduit carries exhaust gas from patient 20 and includes an exhaust valve at the end distal from patient 20. The exhaust valve in such an embodiment is typically actively controlled to maintain a desired level or pressure in the system, which is commonly known as positive end expiratory pressure (PEEP).

Furthermore, in the illustrated exemplary embodiment shown in FIG. 1, patient interface 24 is a nasal/oral mask. It is to be understood, however, that patient interface 24 can include a nasal mask, nasal pillows, or any other device that sealingly engages patient 20 and provides a suitable gas flow communicating function. Also, for purposes of the present invention, the phrase “patient interface” can include delivery conduit 22 and any other structures that couple the source of pressurized breathing gas to patient 20.

In the illustrated embodiment, pressure support system 12 includes a pressure controller in the form of a valve 28 provided in internal delivery conduit 30 provided in a housing of pressure support device 14. Valve 28 controls the pressure of the flow of breathing gas from gas flow generator 16 that is delivered to patient 20. For present purposes, gas flow generator 16 and valve 28 are collectively referred to as a pressure generating system because they act in concert to control the pressure and/or flow of gas delivered to patient 20. However, it should be apparent that other techniques for controlling the pressure of the gas delivered to patient 20, such as varying the blower speed of gas flow generator 16, either alone or in combination with a pressure control valve, are contemplated by the present invention. Thus, valve 28 is optional depending on the technique used to control the pressure of the flow of breathing gas delivered to patient 20. If valve 28 is eliminated, the pressure generating system corresponds to gas flow generator 16 alone, and the pressure of gas in the patient circuit is controlled, for example, by controlling the motor speed of gas flow generator 16.

Pressure support system 12 further includes a flow sensor 32 that measures the flow of the breathing gas within internal delivery conduit 30 and delivery conduit 22. In the particular embodiment shown in FIG. 1, flow sensor 32 is interposed in line with delivery conduits 30 and 22, most preferably downstream of valve 28. Pressure support system 12 additionally includes a pressure sensor 37 that detects the pressure of the pressurized fluid in internal delivery conduit 30. While the point at which the flow is measured by flow sensor 32 and the pressure is measured by pressure sensor 37 are illustrated as being within pressure support device 14, it is to be understood that the location at which the actual flow and pressure measurements are taken may be anywhere along delivery conduits 30 or 22. The flow of breathing gas measured by flow sensor 32 and the pressure detected by pressure sensor 37 are provided to processing unit 34 to determine the flow of gas at patient 20 (QPATIENT).

Techniques for calculating QPATIENT are well known, and take into consideration the pressure drop of the patient circuit, known leaks from the system, i.e., the intentional exhausting of gas from the circuit as indicated by arrow E in FIG. 1, and unknown leaks from the system, such as leaks at the mask/patient interface. The present invention contemplates using any known or hereafter developed technique for calculating leak flow, and using this determination in calculating QPATIENT using measured flow and pressure. Examples of such techniques are taught by U.S. Pat. Nos. 5,148,802; 5,313,937; 5,433,193; 5,632,269; 5,803,065; 6,029,664; 6,539,940; 6,626,175; and 7,011,091, the contents of each of which are incorporated by reference into the present invention.

Of course, other techniques for measuring the respiratory flow of patient 20 are contemplated by the present invention, such as, without limitation, measuring the flow directly at patient 20 or at other locations along delivery conduit 22, measuring patient flow based on the operation of gas flow generator 16, and measuring patient flow using a flow sensor upstream of valve 28.

In some non-limiting embodiments of the disclosed concept, pressure support system 12 also includes a proximal pressure sensor 38 that is in fluid communication with a point along delivery conduit 22. For example, without limitation, proximal pressure sensor 38 may be in fluid communication with a point on delivery conduit 22 near patient interface device 24 via a probe connected between proximal pressure sensor 38 and the point on delivery conduit 22. Proximal pressure sensor 38 facilitates measuring pressure proximate the point on delivery conduit 32 and provides the measured proximal pressure to processing unit 34. It will be appreciated that in some exemplary embodiments, proximal pressure sensor 38 may be omitted.

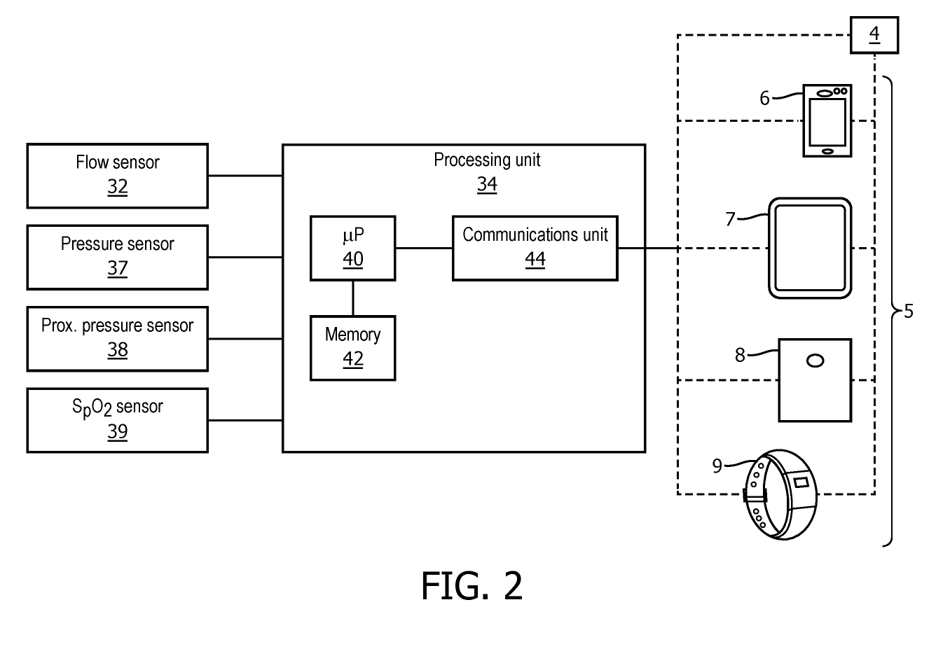

While the flow sensor 32, pressure sensor 37, and proximal pressure sensor 38 have been shown in conjunction with the pressure support system 12 illustrated in FIG. 1, it will be appreciated by those having ordinary skill in the art that other types of sensors may also be employed in conjunction with pressure support system 12 without departing from the scope of the disclosed concept. For example, and without limitation, a temperature sensor may be used to measure temperature, a proximity/contact sensor may be used to sense contact between patient 20 and components of the patient interface. Other types of sensors that may be employed in conjunction with pressure support system include, for example and without limitation, location sensors (e.g, global positioning system sensors) to determine a location of pressure support system 12, light sensors to sense light, an SpO2 sensor (shown in FIG. 2) to detect blood oxygen levels (and to detect reduced CO2 sensitivity (shown in FIG. 2). It will be appreciated that the foregoing examples of types of sensors that may be employed in conjunction with pressure support system 12 is not exhaustive and other types of sensors may also be employed without departing from the scope of the disclosed concept.

Processing unit 34 includes a processing portion which may be, for example, a microprocessor, a microcontroller or some other suitable processing device, and a memory portion that may be internal to the processing portion or operatively coupled to the processing portion and that provides a storage medium for data and software executable by the processing portion for controlling the operation of pressure support system 12. Processing unit 34 is structured to receive outputs of one or more sensors structured to gather data related to effectiveness of the pressure support therapy. An example of such sensors is flow sensor 32 and pressure sensor 37. However, other types of sensors may also gather data related to effectiveness of the pressure support therapy and be employed with processing unit 34, as will be described in more detail herein. Processing unit 34 is also structured to analyze outputs of the sensors while pressure support therapy is provided to the patient to determine patient airflow and pressure waveforms in the patient circuit.

An input/output device 36 is provided for setting various parameters used by airway pressure support system 12, as well as for displaying and outputting information and data to a user, such as a patient, clinician or caregiver.

One or both of an audible indicator 41 (e.g., without limitation, a buzzer) and/or a visual indicator 43 (e.g., a flashing light) which may be selectively activated by processing unit 34 may be provided in or on pressure support device 14.

It will be appreciated that pressure support device 14 may include additional components that are not illustrated in the schematic diagram of FIG. 1. For example, without limitation, pressure support device 14 may include a filter to filter breathing gas provided to patient 20 and a humidifier to humidify breathing gas provided to patient 20.

In the illustrated, non-limiting exemplary embodiment of the present invention, airway pressure support system 12 essentially functions as a CPAP pressure support system and pressure support device 14 provides functions of a CPAP base unit. Pressure support system 12, therefore, includes all of the capabilities necessary in such systems in order to provide appropriate CPAP pressure levels to patient 20. This includes receiving the necessary parameters, via input commands, signals, instructions or other information, for providing appropriate CPAP pressure, such as maximum and minimum CPAP pressure settings. It should be understood that this is meant to be exemplary only, and that other pressure support methodologies, including, but not limited to, BiPAP® AutoSV, AVAPS, Auto CPAP, and BiPAP Auto, are within the scope of the present invention.

FIG. 2 is a block diagram of processing unit 34 in accordance with a non-limiting exemplary embodiment of the disclosed concept. Processing unit 34 includes a processor 40, a memory 42, and a communication unit 44. Processor 40 may form all or part of a processing portion which may be, for example, a microprocessor, a microcontroller or some other suitable processing device. Memory 42 may form all or part of a memory portion that may be internal to the processing portion or operatively coupled to the processing portion and provide a storage medium for data and software executable by the processing portion for implementing functionality of processing unit 34 and controlling the operation of pressure support system 12. Memory 42 can be any of one or more of a variety of types of internal and/or external storage media such as, without limitation, RAM, ROM, EPROM(s), EEPROM(s), FLASH, and the like that provide a storage register, i.e., a machine readable medium, for data storage such as in the fashion of an internal storage area of a computer, and can be volatile memory or nonvolatile memory.

Communication unit 44 may provide for communication between processing unit 34 and other components of pressure support device 14, components of the patient circuit, or other external devices, such as central processing unit 4, via the internet, cellular, WiFi, wired telephone line, or any other suitable means. For example, without limitation, communication unit 44 may facilitate communication with various sensors such as flow control sensor 32. Communication unit 44 may also facilitate communication with external devices. For example, without limitation, communication unit 44 may facilitate communication with electronic devices such as a phone, tablet, computer, or other devices whether local or distant, directly or via a network and may further communicate information from local device to central processing unit 4. Communication facilitated by communication unit 44 may allow processing unit 34 to send and/or receive data from the component or device it communicates with.

As previously described, processing unit 34 receives outputs from one or more sensors such as, for example and without limitation, flow sensor 32, pressure sensor 37, and SpO2 sensor 39. Although flow sensor 32, pressure sensor 37, and SpO2 sensor 39 are shown, it will be appreciated that one or more of these sensors may be omitted without departing from the scope of the disclosed concept. It will also be appreciated that processing unit 34 may receive outputs from one or more other types of sensors that are not shown in FIG. 2. For example, without limitation, processing unit 34 may receive outputs from one or more of a temperature sensor, a proximity/contact sensor, location sensors, microphones, or identification sensors.

Referring to FIG. 2, system 2 may include one or more input devices 5, which may be in communication (either directly or indirectly depending on the application) with one or both of processing unit 34 and/or central processing unit 4 via any suitable means (e.g., Bluetooth, Wifi, cellular, etc.) Input devices may include, for example, without limitation a smartphone device 6, an electronic tablet 7, a smart scale 8 and a smart watch 9. Input devices 5 may be utilized to supply a variety of data regarding details of the patient. For example, smartphone device 6 and/or electronic tablet 7 may be utilized by the patient in responding to questions regarding the patient's health, eating habits, exercise habits, perceived sleep quality, etc. Smartphone device 6 and/or electronic tablet 7 may also be utilized to provide visual or audio data regarding the patient such as, for example without limitation, images of the patient's face which may be utilized to monitor for changes in facial air or facial geometry, audio clips of the patient sleeping, etc. As another example, smart scale 8 may provide data regarding the patient's weight and smart watch 9 may provide data regarding the patient's activity levels, heart rate, etc.

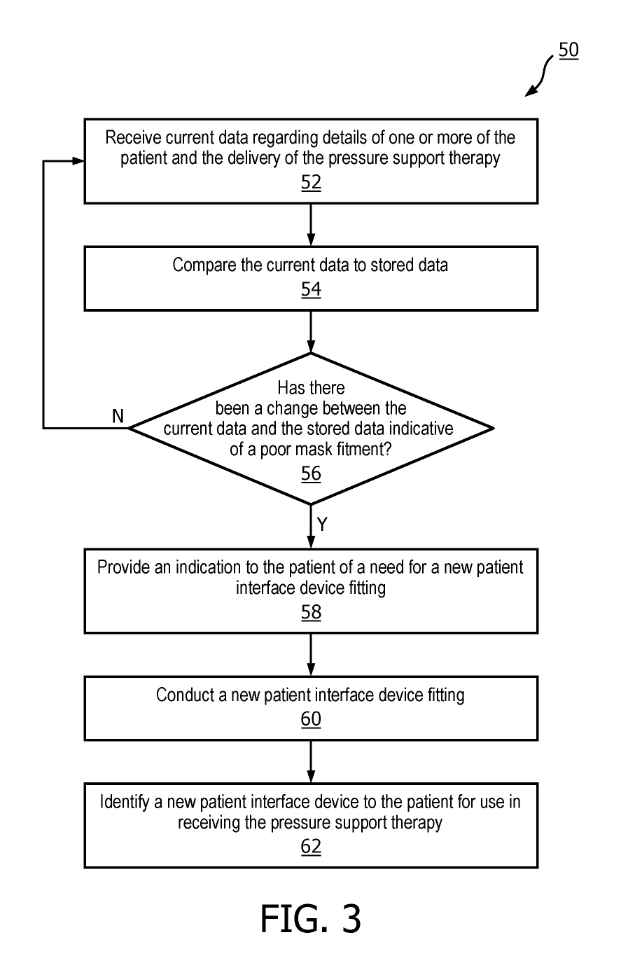

Having thus described the basic components of system 2 and the functionality thereof, an example method 50 for improving delivery of a pressure support therapy provided by pressure support system 12 according to one particular, non-limiting exemplary embodiment of the present invention will now be described in conjunction with the flow chart of FIG. 3. Method 50 begins at 52 wherein current data regarding details of one or more of patient 20 and the delivery of the pressure support therapy are received by one or both of central processing unit 4 (if method 50 is generally carried out distantly) and/or processing unit 34 (if method 34 is generally carried out locally). For example, such data may include details of the pressure support therapy provided by one or more sensors associated therewith such as previously discussed. Such details may include, for example, without limitation, a leak score of the patient interface device, therapy pressure(s) delivered to the patient, breathing rate, large leak minutes, therapy time (indication of compliance), and CPAP Auto pressure. Increases in leak score might indicate that a facial geometry has changed. Increased CPAP therapy pressure might be an indicator that the disease state of the patient is worsening or that greater leak is present, which both may lend itself to the creation of a new custom patient interface. It is also to be appreciated that aside from the aforemetioned metrics that there are a number of other metrics that can be taken from the pressure support therapy and used to help diagnose potential lapses in therapy due to poor fitment of patient interface device 24.

As another example, such data received at 52 may include details of the patient such as recorded and/or communicated via one or more of input devices 5. Such details of the patient may include, for example, without limitation, heart rate, blood pressure, pulse oximetry, glucose levels, body temperature, body weight, recent activity of the patient, facial images of the patient, and perceived level of sleepiness. Changes in weight may change the facial geometry of the patient and/or cause differences in the hardness/softness thereof, which could affect mask size/fit. Increased sleepiness may indicate that the patient is not receiving the proper therapy and would benefit from a new custom cushion. Detection of changes in facial hair may dictate the type of CPAP mask that would offer the best therapy. Alternative secondary metrics obtained from the patient can be surveys that are taken by the patient that relate to their potential satisfaction with the fit or comfort of a mask over time. By collecting this information there can be potential to diagnose non-fit characteristis of a mask promoting further scans for changes in the facial geometry of the patient.

Next, as shown at 54, the current data received in 52 is compared by central processing unit 4 or processing unit 34 to stored data from one or more previous pressure support therapies contained in the associated memory thereof. Next, as shown at 56, if it is determined that there has been a change between the current data and the stored data indicative of a poor mask fitment, an indication is provided to the patient and/or to a caregiver thereof (e.g., a doctor, nurse, etc.) that the patient is likely in need of a new patient interface device fitting, such as shown at 58. Such indication may be provided, for example, without limitation, via an indicator provided on pressure support device 14, by a communication (e.g., phone call, text, ping, etc.) sent via one or more of input devices 5, or via any other suitable means.

Next, as shown at 60, a new patient interface device fitting is carried via any suitable means (e.g., facial imaging, test fitments, etc.) in which a new patient interface device which would provide for improved delivery of the pressure support therapy to the patient is determined.

Then, as shown at 62, the new patient interface device is then identified and/or provided to the person. As an example, the new patient interface device may be identified to the person via information provided to the user, via any suitable form (e.g., electronically or via hardcopy), particularly specifying the patient interface device (i.e., specifications which particularly identify the device either from amongst other devices or how to construct from scratch or from components). For example, without limitation, a prescription for obtaining a particular interface device or a CAD file or similar item containing instructions and/or dimensional information for constructing a custom interface device may be provided. Alternatively, the interface device may be identified to the person by providing the person with the actual device, be it custom-made or an off-the-shelf item. In the case of a custom-made device, a 3-D printer or other suitable automated manufacturing device may be used to provide the device to the patient either generally immediately subsequent to the device fitting or at a later time (i.e., via special delivery, pick-up from a special location, etc.).

From the foregoing it is thus to be appreciated that embodiments of the present invention utilize a holistic approach in considering numerous characteristics of the patient and/or the therapy delivered to the patient in order to identify potential items which may indicate changes are available which may improve the therapy.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method of improving delivery of a pressure support therapy provided by a pressure support device to a patient via a patient interface device, the method comprising:
receiving current data regarding details of one or more of the patient and the delivery of the pressure support therapy;
comparing the current data to stored data;
determining a change between the current data and the stored data indicative of a poor mask fitment; and
providing an indication of a need for a new patient interface device fitting.

2. The method of claim 1, further comprising conducting a new patient interface device fitting with the patient and identifying a new patient interface device for the patient.

3. The method of claim 2, wherein identifying the new patient interface device for the patient comprises providing the patient with a specification of the new patient interface device.

4. The method of claim 2, wherein identifying the new patient interface device for the patient comprises providing the patient with the new patient interface device.

5. The method of claim 1, wherein receiving details regarding one or more of the patient and the delivery of the pressure support therapy comprises receiving details regarding a leak score of the patient interface device.

6. The method of claim 1, wherein receiving details regarding one or more of the patient and the delivery of the pressure support therapy comprises receiving details regarding therapy pressure provided during the pressure support therapy.

7. The method of claim 1, wherein receiving details regarding one or more of the patient and the delivery of the pressure support therapy comprises receiving details regarding one or more of: weight, heart rate, blood pressure, pulse oxsimetry, glucose level, body temperature, or recent activity of the patient.

8. The method of claim 1, wherein receiving details regarding one or more of the patient and the delivery of the pressure support therapy comprises receiving details regarding sleepiness of the patient.

9. The method of claim 1, wherein receiving details regarding one or more of the patient and the delivery of the pressure support therapy comprises receiving details regarding changes in facial hair of the patient.

10. The method of claim 1, wherein receiving details regarding one or more of the patient and the delivery of the pressure support therapy comprises receiving details provided by the user in the form of responses to questions.

11. The method of claim 10, wherein the responses to questions are provided electronically.

12. The method of claim 1, wherein receiving details regarding one or more of the patient and the delivery of the pressure support therapy comprises receiving details regarding the patient in the form of images of the patient.

13. The method of claim 1, wherein receiving details regarding one or more of the patient and the delivery of the pressure support therapy comprises receiving details of the pressure support therapy sent by a communications device which is part of the pressure generating device.

14. A system for use in improving delivery of a pressure support therapy provided to a patient via a patient interface device, the system comprising:
a pressure support system having a pressure support device;
a processing unit; and
a number of input devices, wherein the processing unit is programmed to:
receive current data regarding details of one or more of the patient and the delivery of the pressure support therapy from one or more of: the number of input devices and a number of sensors associated with the pressure support device;
compare the current data to stored data;
determine a change between the current data and the stored data indicative of a poor mask fitment; and
provide an indication of a need for a new patient interface device fitting.

15. The system of claim 14, wherein the processing unit is a component of the pressure support device.

16. The system of claim 14, wherein the processing unit is remotely located from the pressure support system.
